(19) United States
(12) Patent Application Publication
(10) Pub. No.: US 2022/0202313 A1
(43) Pub. Date: Jun. 30, 2022

(54) METHOD FOR ESTIMATING A HEART RATE OR A BREATHING RATE

(71) Applicant: COMMISSARIAT A L'ENERGIE ATOMIQUE ET AUX ENERGIES ALTERNATIVES, Paris (FR)

(72) Inventor: Stéphane BONNET, Grenoble cedex (FR)

(73) Assignee: COMMISSARIAT A L'ENERGIE ATOMIQUE ET AUX ENERGIES ALTERNATIVES, Paris (FR)

(21) Appl. No.: 17/645,852

(22) Filed: Dec. 23, 2021

(30) Foreign Application Priority Data
Dec. 26, 2020 (FR) 2014157

(51) Int. Cl. A61B 5/08; A61B 5/024; A61B 5/00

(52) U.S. Cl. CPC A61B 5/0816; A61B 5/7225; A61B 5/024

(57) ABSTRACT

A method for determining a frequency or period of a time-domain variation in a physiological parameter, the physiological parameter varying periodically as a function of time, under the effect of a cardiac activity or respiratory activity of a user, the method comprising the following steps:
a) detecting, using a detector, a signal representative of the physiological parameter of the user at various times;
b) on the basis of the detected signals, obtaining a measurement function (g);
c) obtaining a pulsed component (g_{AC}) of the measurement function, the pulsed component being a periodic function the period of which depends on the cardiac activity or respiratory activity;
d) on the basis of the pulsed component, determining heart rate (HR) or breathing rate (RR);

the method being characterized in that step d) comprises selecting characteristic times of the pulsed component.

### TECHNICAL FIELD

The technical field of the invention is estimation of a heart rate or of a breathing rate.

PRIOR ART

Photoplethysmography (PPG) is a non-invasive optical method that allows variations in the volume of blood in surface tissues to be evaluated through the variation of the absorption of light in these tissues. This method allows physiological parameters, such as heart rate or degree of oxygenation, to be estimated. It is based on a measurement of variations in the light transmitted or backscattered by the tissues using an optical sensor.

One example of implementation of PPG is pulse oximetry, which is carried out using a clip that is fastened to a finger or to the lobe of an ear. This type of device comprises a light source and a photodetector, the finger or lobe being placed between the light source and the photodetector, in a transmission configuration. In other applications of PPG, the light source and the photodetector are located side-by-side. This is for example the case of the PPG devices integrated into watches. The photodetector detects photons backscattered by the tissues illuminated by the light source. This is what is called a backscattering configuration.

Whatever the configuration, the signal detected by the photodetector comprises a continuous component, to which is added a pulsed component, the latter varying as a function of heart rate. The information relative to heart rate is contained in the pulsed component. The latter may be extracted by carrying out high-pass filtering on the signal detected by the photodetector.

Amplitude thresholding, applied to the pulsed component, allows characteristic times, typically extrema (maxima or minima), to be identified. The time interval separating the characteristic times allows a heart rate to be estimated. One example of application is for example given in U.S. Pat. No. 9,778,111B2.

However, the pulsed component of a PPG optical signal may contain large intensity fluctuations. Application of intensity thresholds to select the characteristic times may result in a lack of accuracy.

The inventor provides a method, which is simple to implement and which does not involve intensity thresholding, for determining heart rate, or breathing rate, or another periodic physiological parameter, on the basis of non-invasive measurements.

### SUMMARY OF THE INVENTION

A first subject of the invention is a method for determining a frequency or period of a time-domain variation in a physiological parameter, the physiological parameter varying periodically as a function of time, under the effect of a cardiac activity or respiratory activity of a user, the method comprising the following steps:
a) detecting, using a detector, a signal representative of the physiological parameter of the user at various times;
b) on the basis of the detected signals, obtaining a measurement function;
c) obtaining a pulsed component of the measurement function, the pulsed component being a periodic function the period of which depends on the cardiac activity or respiratory activity;
d) on the basis of the pulsed component, determining heart rate or breathing rate; the method being characterized in that step d) comprises:
   di) obtaining a frequency-domain expression for the pulsed component;
   dii) selecting a dominant frequency of the frequency-domain expression;
   diii) filtering the frequency-domain expression at the dominant frequency, so as to obtain a filtered frequency-domain expression;
   div) on the basis of the filtered frequency-domain expression, obtaining a sinusoidal time-domain function having the dominant frequency;
   dv) taking into account a selection condition and selecting characteristic times of the sinusoidal time-domain function resulting from div), each characteristic time corresponding to a time at which said sinusoidal time-domain function meets the selection condition;
   dvi) on the basis of the characteristic times of the sinusoidal time-domain function, selecting characteristic times of the pulsed component, each characteristic time of the pulsed component corresponding to a time at which said pulsed component meets the selection condition;
   dvii) determining the frequency or period of the time-domain variation in the physiological parameter on the basis of the characteristic times of the pulsed component resulting from dvi).

According to one embodiment:
diii) comprises carrying out bandpass filtering on the frequency-domain expression resulting from di), in a passband containing the dominant frequency selected in dii), so as to obtain a filtered frequency-domain expression;
div) comprises computing a time-domain expression for the filtered frequency-domain expression, the computed time-domain expression corresponding to the sinusoidal function.

According to one possibility:
in dv), the selection condition is reaching an extremum, the extremum either being a maximum, or a minimum, each characteristic time being one extremum of the sinusoidal function;
in dvi), each characteristic time is one extremum of the pulsed component.

According to another possibility, the selection condition may be a passage through a predetermined value, for example a passage through zero.

Step dvi) may comprise:
defining a time interval about each characteristic time selected on the basis of the sinusoidal time-domain function, the time interval being shorter than one period of the sinusoidal time-domain function;
in each time interval, selecting a time at which the pulsed component meets the selection condition.

According to one embodiment:
step div) implements a dynamic-time-warping algorithm, so as to optimize a match between the sinusoidal function and the pulsed component;
in dvi), the characteristic times of the pulsed component correspond to the characteristic times of the sinusoidal function.

According to one embodiment, dvii) comprises estimating a heart rate or period. The heart rate or heart period may be estimated on the basis of lengths of time separating successive characteristic times of the pulsed component. Step dvii) may comprise:
computing a time-domain variation in the heart rate or period;
determining at least one period of the time-domain variation in the heart rate or period;
estimating a breathing rate or period on the basis of the period of said time-domain variation.

Step dvii) may comprise applying smoothing to the time-domain variation in the heart rate, prior to determining the breathing rate or period.

According to one embodiment, the method may then comprise a step of characterizing cardiac or respiratory activity, comprising:
forming a baseline, passing through the time-domain measurement function or the pulsed component at each characteristic time resulting from dvi);
obtaining a characterization function, by subtracting the time-domain measurement function or the pulsed component from the baseline, the characterization function comprising lobes, each lobe extending between two successive characteristic times;
characterizing cardiac or respiratory activity on the basis of a shape or of an area of the lobes of the characterization function.

Advantageously, according to this embodiment, the selection condition taken into account in dv) and dvi) is reaching an extremum.

According to one embodiment:
in step a), the detector is a photodetector;
step a) comprises applying an optical device against a bodily region (2) of the user, the optical device comprising:
   a light source arranged to emit light toward the bodily region;
   the photodetector, arranged to detect an intensity of light emitted by the light source and having propagated through the bodily region;
step a) comprises illuminating the bodily region with the light source and measuring the intensity detected by the photodetector at various times;
in step b), the measurement function corresponds to a time-domain variation in detected intensity.

According to one embodiment:
in step a), the detector is an acoustic detector;
step a) comprises placing an acoustic device against a bodily region of the user, the acoustic device comprising:
   an acoustic source, arranged to emit an ultrasonic wave toward the bodily region;
   the acoustic detector, arranged to detect an acoustic wave reflected by the bodily region;
step a) comprises insonifying (exposing to ultrasound) the bodily region using the acoustic source and measuring the acoustic wave reflected by the bodily region at various times;
in step b), the measurement function corresponds to a time-domain variation in the measured acoustic wave.

According to one embodiment:
in step a), the detector is a pressure detector;
step a) comprises placing the pressure detector against a bodily region of the user;
step a) comprises measuring the pressure exerted by the bodily region at various times;
in step b), the measurement function corresponds to a time-domain variation in the measured pressure.

A second subject of the invention is a device for determining a frequency or period of a time-domain variation in a physiological parameter, the physiological parameter varying periodically as a function of time, under the effect of a cardiac activity or respiratory activity of a user, the device comprising:
a detector, configured to measure a signal representative of a physiological parameter of the user at various times;
a processing unit, configured to implement steps b) to d) of a method according to the first subject of the invention, on the basis of signals detected by the detector at the various times.

The device may comprise a light source, arranged to emit light toward a bodily region of the user, the detector being a photodetector, arranged to detect an intensity of light emitted by the light source and having propagated through the bodily region.

The device may comprise an acoustic source, arranged to emit an ultrasonic wave toward a bodily region of the user, the detector being an acoustic detector, arranged to detect an acoustic wave reflected by the bodily region.

The detector may be a pressure sensor, arranged to measure a pressure exerted by the bodily region.

The invention will be better understood on reading the description of the examples of embodiment, which are described, in the rest of the description, with reference to the figures listed below.

### FIGURES

FIGS. 1A and 1B schematically show a first embodiment of a device according to the invention.

FIG. 1C shows another embodiment of a device according to the invention.

FIG. 2A shows an example of PPG signals detected in three spectral bands, respectively.

FIG. 2B is a detail of FIG. 2A.

FIG. 3 schematically shows the main steps of a method according to the invention.

FIG. 4A shows the pulsed components of the PPG signals shown in FIG. 2B.

FIG. 4B is a detail of FIG. 4A.

FIG. 4C is a frequency-domain expression for pulsed components, such as shown in FIGS. 4A and 4B, each expression being computed in a time window corresponding to 256 samples.

FIG. 4D shows a pulsed component of the PPG signal, i.e. a component such as shown in FIG. 4A, and an obtained sinusoidal function having a dominant frequency of the pulsed component.

FIG. 4E is a detail of FIG. 4D. FIG. 4E illustrates a search for maxima in the pulsed component on the basis of maxima of the sinusoidal function.

FIG. 5A shows a time-domain variation in a heart rate estimated by implementing the steps schematically shown in FIG. 3, on the basis of a PPG signal as shown in FIG. 2A.

FIG. 5B is a detail of FIG. 5A.

FIG. 5C was obtained by smoothing FIG. 5A.

FIG. 5D is a detail of FIG. 5C.

FIG. 6A shows a sinusoidal function obtained by dynamic time warping.

FIG. 6B corresponds to FIG. 5B.

FIG. 6C shows a time-domain variation in a heart rate, determined on the basis of FIG. 6A, in a time interval identical to that considered with respect to FIG. 6B.

FIG. 7A shows a PPG signal on which characteristic times have been indicated. A baseline joining the PPG signal between the characteristic times has also been shown (dashed line).

FIG. 7B shows a characterization function obtained on the basis of the PPG signal and the baseline shown in FIG. 7A.

FIG. 7C is a detail of FIG. 7B.

### DESCRIPTION OF PARTICULAR EMBODIMENTS

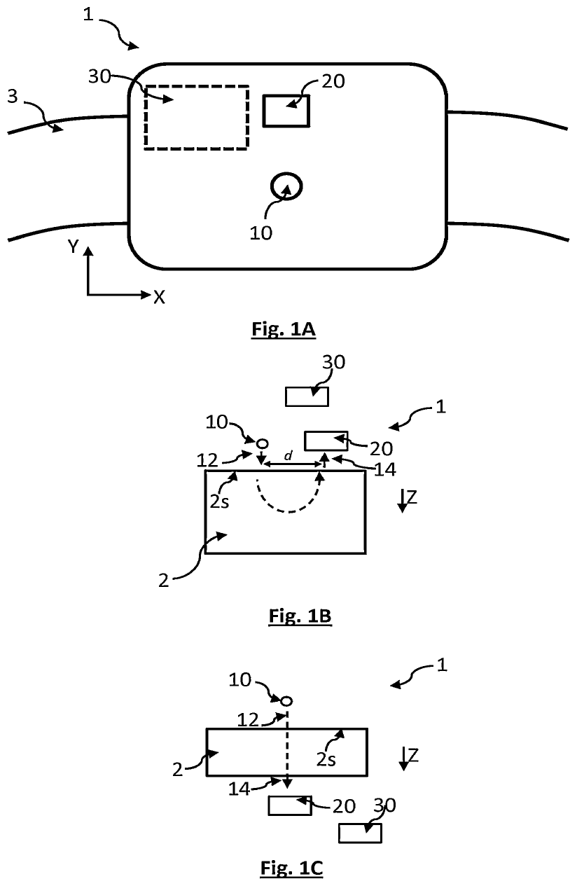

FIG. 1A shows an example of a device 1 allowing the invention to be implemented. The device comprises a detector 20, configured to detect a signal representative of a physiological parameter of a user at various times. The user may be a living animal or human being. The physiological parameter measured by the detector allows a heart rate or a breathing rate to be estimated. In the embodiment described with reference to FIGS. 1A to 1C, the device 1 is an optical device. In another embodiment, the device 1 may be an acoustic device.

The optical device 1 is intended to be applied against a bodily region 2 of the user. The bodily region may for example be located on a wrist or finger of the user. In the example shown, the device 1 is connected to a fastening 3 forming a watch strap, so as to be fastened against a wrist. The device may be applied against any other sufficiently vascularized bodily region: stomach, chest, earlobe, elbow, finger, leg, these examples being nonlimiting.

The device 1 comprises a light source 10, configured to emit an incident light beam 12 toward the bodily region 2 facing which the light source is placed. The incident light beam 12 propagates toward the bodily region 2 along a propagation axis Z. The photons of the incident light beam 12 penetrate into the bodily region 2 and some of said photons are backscattered, for example in a direction parallel to the propagation axis Z, back the way they came. The backscattered photons form backscattered radiation 14. The backscattered radiation 14 may be detected by a photodetector 20 placed facing a surface 2s of the bodily region. The photodetector 20 may be configured so as to detect backscattered radiation emanating from the bodily region at a distance d, called the backscatter distance, which is generally nonzero and smaller than a few millimeters, typically smaller than 15 mm or 10 mm. The photodetector 20 allows the intensity of the backscattered radiation to be measured.

The light source 10 may be a light-emitting diode (LED), the emission spectral band of which lies in the visible or in the infrared. Preferably, the width of the emission spectral band is narrower than 100 nm. The photodetector 20 may be a photodiode. The signal detected by the photodetector is a photoplethysmography (PPG) signal.

FIG. 1B schematically shows the main components of the device 1 in a plane perpendicular to the surface 2s of the bodily region. The curved dashed arrow shows an optical path of photons emitted by the light source, in the analyzed bodily region.

The optical device 1 comprises a processing unit 30 configured to process a signal detected by the photodetector 20. The processing unit 30 is connected to a memory 32, in which instructions for implementing the method described below are stored. The processing unit may comprise a microprocessor.

According to one alternative, shown in FIG. 1C, the bodily region 2 lies between the light source 10 and the photodetector 20. In such a configuration, which is called the transmission configuration, the photodetector 20 measures an intensity of the photons having passed through the bodily region 2. Such a configuration assumes that the bodily region is sufficiently thin for a sufficient quantity of photons to emanate 30 from the medium and to be detected by the photodetector. It may for example be a question of an earlobe or of a finger.

Whatever the embodiment, the photodetector 20 is arranged to measure an intensity of a light beam formed by photons that have propagated through the bodily region 2: it is a question either of backscattered photons, or of photons having passed through the bodily region. In the rest of the description, the reflective configuration shown in FIG. 1B will be considered, the bodily region being a finger of a patient.

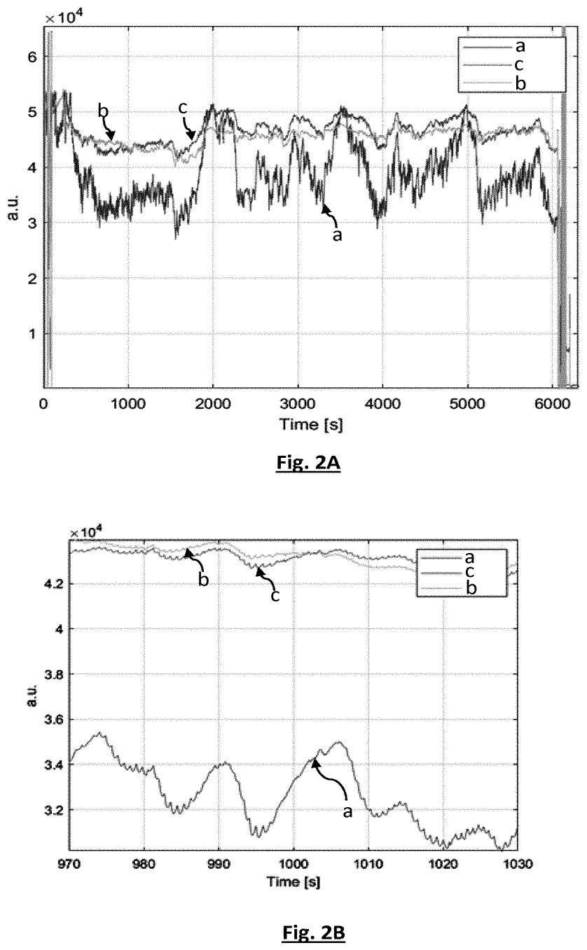

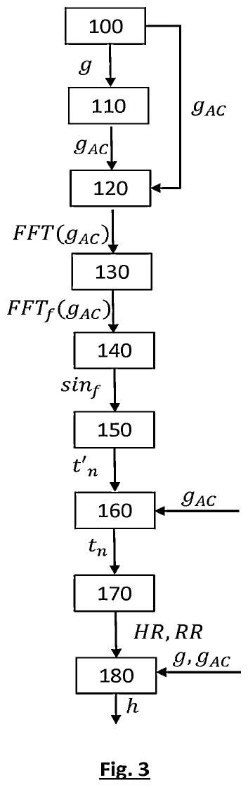

FIGS. 2A and 2B show examples of signals PPG detected, in reflection, by the photodetector following illumination of a bodily region (finger) in three spectral bands centered on the green (curve a−γ=574 nm), the red (curve b−γ=645 nm) and the infrared (curve c−γ=940 nm), respectively. FIG. 2A shows a measurement function g, corresponding to the signal PPG measured for 6000 seconds. FIG. 2B is a detail of FIG. 2A, in a narrower time range, between 970 s and 1030 s. In FIGS. 2A and 2B, the x-axis represents time (s) and the y-axis is the detected light intensity (arbitrary units). Measurement function designates the signals measured by the photodetector at various measurement times.

FIG. 3 schematically shows the main steps allowing an inter-beat interval (or heart period) that may be converted into a heart rate HR to be estimated. The method com prises:
extracting a pulsed component g_{AC} of the measurement function g: the pulsed component is a periodic function the period of which depends on heart rate;
estimating the heart rate on the basis of the extracted pulsed component, without intensity thresholding of the pulsed component.

The main steps of the method will now be described.

Step 100: acquiring the signal PPG. It is a question of acquiring a signal detected by the photodetector 20 during a determined time range. A measurement function g corresponding to the signal PPG, such as shown in FIGS. 2A and 2B, is obtained. The acquisition frequency is preferably higher than 10 Hz, and for example 50 Hz.

During the acquisition, the low-frequency component of the measurement function g may be removed by applying high-pass analog filtering, so as to remove frequency components of the signal below a cut-off frequency, the latter preferably being lower than 0.5 Hz, and for example 0.2 Hz. In this case, the signal output by the detector represents the pulsed component g_{AC} of the PPG signal directly.

Step 110: when the high-pass filtering is not carried out in an analog manner during the acquisition, step 110 is implemented, in the processing unit 30, so as to extract a pulsed component from the measurement function g. This step amounts to applying a high-pass digital filter. It may be implemented by subtracting, from the measurement function g, at a time t, an average of the measurement function over a predetermined duration, 1 second for example, and centered on the time t:

g_{AC} (t)=g (t)−g̅(t) (1)

g̅(t) is an average of g(t) computed in a time interval comprising the time t, and preferably centered on the time t.

Step 110 may be implemented with other digital filtering techniques, for example a finite-impulse-response filter, a Savitzky-Golay filter for example, or even an infinite-impulse-response filter. It allows the pulsed component g_{AC} to be extracted from the measurement function g detected by the photodetector 20.

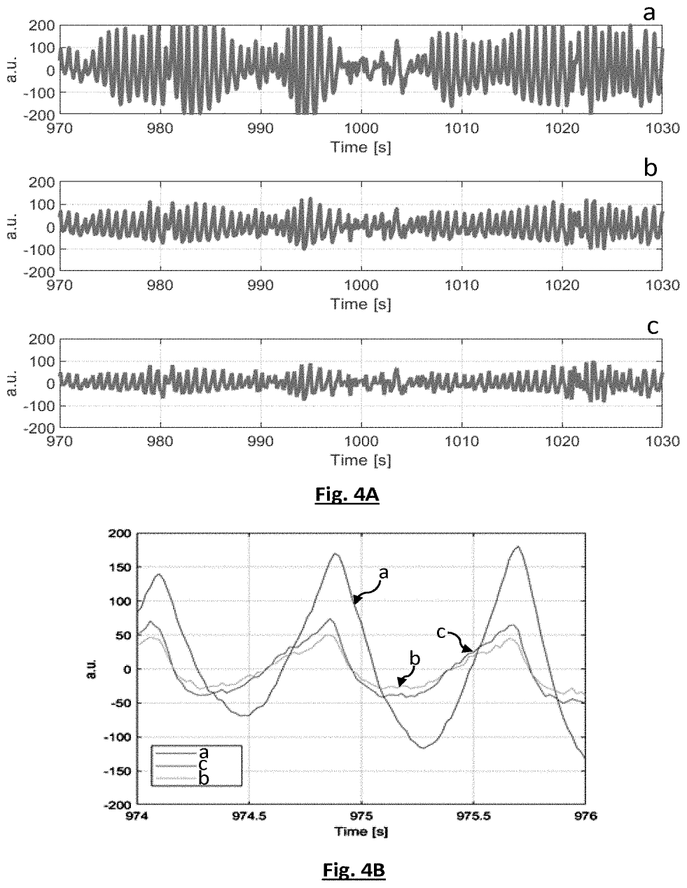

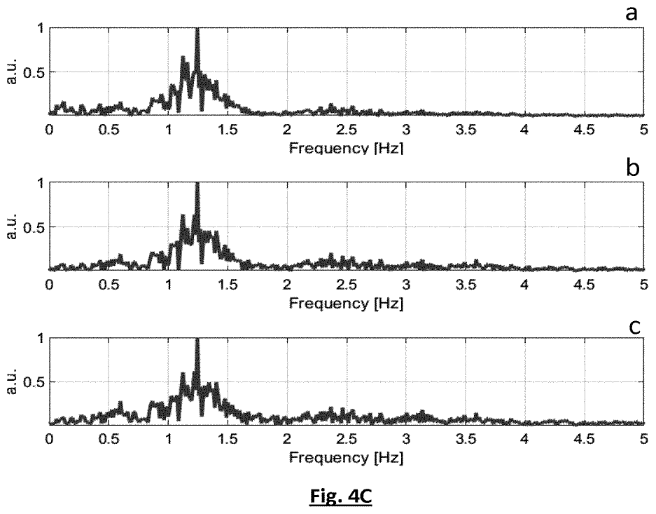

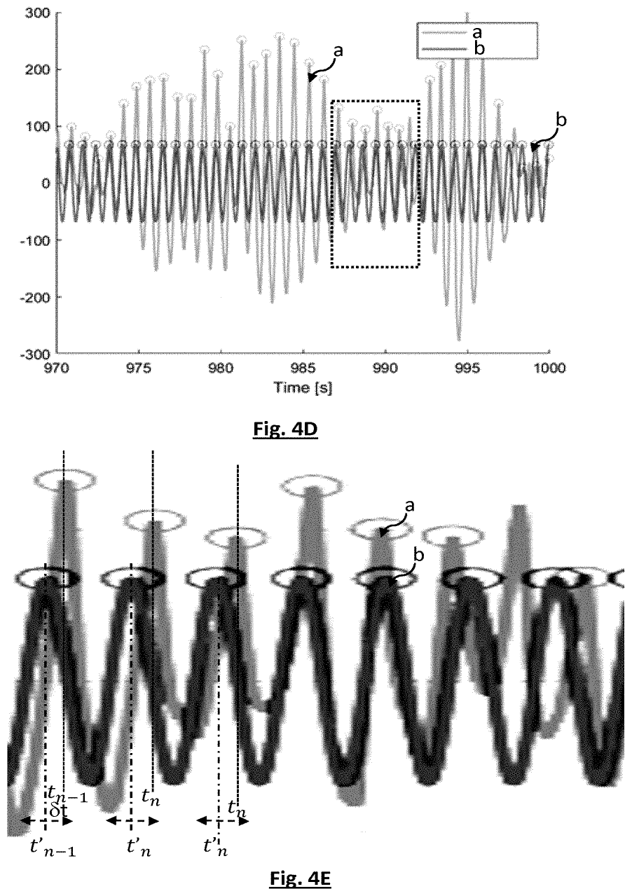

FIG. 4A shows a pulsed component g_{AC} extracted from the measurement function g shown in FIG. 2B. In FIG. 4A, the measurement functions have been shown in the three spectral bands mentioned above: the green (curve a−λ=574 nm), the red (curve b−γ=645 nm) and the infrared (curve c−γ=940 nm). FIG. 4B is a detail of the curves shown in FIG. 4A, in a narrower time range. It may be seen that the pulsed components extracted from measurement functions g formed in the three spectral bands are periodic functions, each period corresponding to one heartbeat. The three components shown in FIGS. 4A and 4B may be used to determine heart rate. However, in the green spectral band, the dynamic range is higher than in the red and infrared spectral bands. The green spectral band is therefore preferred. In the rest of the description, consideration will be limited to the component acquired in the green spectral band; however, the method may be applied in the other spectral bands, in the visible or infrared domain.

The pulsed component g_{AC} is a periodic function, the period of which depends on heart rate. Each maximum of the pulsed component g_{AC} corresponds to a time at which the volume of blood, in the illuminated bodily region, is minimum. The decrease following each maximum corresponds to the cardiac systole.

Step 120: frequency-domain expression for the pulsed component.

In step 120, a Fourier transform is applied to the pulsed component g_{AC}. This allows a frequency-domain expression FFT(g_{AC}) for the pulsed component g_{AC} to be obtained. The Fourier transform is computed by applying a moving time window At of a few seconds, for example of a little more than 5 seconds, i.e. of 256 samples if the acquisition frequency of 50 Hz mentioned in step 100 is taken into account. Other types of transforms allowing a frequency-domain expression for g to be obtained are envisionable.

FIG. 4C shows a frequency-domain expression FFT(g_{AC}) computed, in a time window of 256 samples, on the basis of functions such as illustrated in FIGS. 4A and 4B—curve a: γ=574 nm; curve b: γ=645 nm; curve c: γ=940 nm. In FIG. 4C, the x-axis corresponds to frequency (units Hz) and the y-axis corresponds to spectral power (arbitrary units).

Step 130: selecting a dominant frequency.

In step 130, the frequency at which the spectral power f of the frequency-domain expression FFT(g_{AC}) is maximum is selected. The selected frequency is a dominant frequency. The other frequencies are set to zero. Thus, a filtered frequency-domain expression is obtained at the dominant frequency, i.e. the expression is restricted to the dominant frequency.

Step 140: computing a sinusoidal time-domain function on the basis of the selected dominant frequency.

In this step, an inverse Fourier transform is applied to the frequency-domain expression FFT_{f}(g_{AC}), which is restricted to the dominant frequency f resulting from step 130. A time-domain sinusoidal function sin_{f}(t)=FFT⁻¹_{f}(g_{AC}), which is aligned with the pulsed component g_{AC}, is obtained. By aligned, what is meant is that, to within an uncertainty, the maxima and minima of the pulsed component g_{AC} are in temporal alignment with the maxima and minima of the time-domain sinusoidal function sin_{f}.

FIG. 4D shows the pulsed component g_{AC} (in gray—curve a) and the sinusoidal function sin_{f} (in black—curve b). FIG. 4E is a detail of FIG. 4D, corresponding to the dashed box drawn in FIG. 4D. In FIGS. 4D and 4E, the x-axis represents time and the y-axis the value of each function. The maxima of each function have been encircled.

It may be seen that the minima and maxima of the pulsed component g_{AC}, of the sinusoidal function sin_{f} are roughly in temporal alignment but appear with a slight temporal offset. FIG. 4E allows certain temporal offsets to be identified. It may also be seen that the offset is not constant. The pulsed component is sometimes “in advance” with respect to the sinusoidal function, and sometimes behind. There is therefore a certain time-domain phase shift between the sinusoidal function and the pulsed component g_{AC}.

As may be seen in FIGS. 4D and 4E, there is a variable time-domain phase shift between the sinusoidal function sin_{f}(t) and the pulsed component g_{AC}(t). The time-domain phase shift results from the fact that the heart rate HR is not constant but fluctuates over time. This effect is usually designated respiratory sinus arrhythmia (RSA): heart rate tends to increase during an inhalation, and to decrease during an exhalation.

Step 150: selecting characteristic times.

In this step, characteristic times t′ₙ of the sinusoidal function sin_{f}(t) are selected, on the basis of which times characteristic times tₙ, of the pulsed component are selected g_{AC}(t). The characteristic times meet a predetermined selection condition. In this example, the selection condition is that the characteristic time corresponds to a local maximum. According to other possibilities, the selection condition is that the characteristic time passes through a local minimum or passes through a predetermined value, for example 0. The index n is an integer assigned chronologically to each chronological time t′ₙ or tₙ.

It is easy to determine times t′ₙ corresponding to maxima (or minima) of the sinusoidal function sin_{f}. In step 150, on the basis of each time t′ₙ selected on the sinusoidal function sin_{f}, a characteristic time tₙ is selected on the pulsed component g_{AC}, this time meeting the selection condition, in the present case correspondence to a local maximum.

One important aspect of the invention is that advantage is taken of the sinusoidal function sin_{f}, of frequency f, resulting from step 140, to select the characteristic times on the pulsed component g_{AC}. Each maximum of the sinusoidal function is considered to be close to a maximum of the pulsed component g_{AC}. In step 150, a time interval δt is defined around each characteristic time t′ₙ corresponding to a maximum of the sinusoidal function sin_{f}. Each time interval δt has been illustrated by a double-headed arrow in FIG. 4E. A local maximum of the pulsed component g_{AC} is sought in each time interval δt. It may be seen that the local maximum of the pulsed component g_{AC} is located at a time tₙ before or after the time t′ₙ of the local maximum of the sinusoidal component sin_{f}.

The duration of each time interval δt is either set, or configurable, depending on the frequency f of the sinusoidal function sin_{f}. The duration of each time interval δt is shorter than the period

$$\frac{1}{f}$$

and preferably than the half-period

$$\frac{1}{2 f}$$

of the sinusoidal function sin_{f}. For example, the duration of each time interval δt is

$$\frac{2}{3 f} ,$$

this corresponding to a number of measured intensities equal to round

$$( \frac{2 N}{3 \times f} ) ,$$

N being the number of samples during 1 second, which corresponds to the sampling frequency, i.e. to 50, and round designating the operator that returns the “closest integer”.

The sinusoidal function sin_{f} defines an average frequency f, in the moving window Δt taken into account in step 120 to compute the frequency-domain expression FFT(g_{AC}). The characteristic times t′ₙ selected on the sinusoidal function sin_{f} are regularly distributed at the average frequency.

Step 160:

In step 160, the selected characteristic times tₙ are used to determine, at each time tₙ, an inter-beat interval IBI(tₙ) (which corresponds to one heart period) with:

$$I B I ( {t}_{n} ) = {t}_{n + 1} - {t}_{n} \qquad ( 2 )$$

The heart rate HR corresponds to

$$\frac{1}{I B I ( {t}_{n} )}$$

Step 170:

In step 170, the time-domain function HR(t) corresponding to the time-domain variation in the heart rate may be subjected to smoothing, for example by means of a median filter centered on each time tₙ and extending over three successive times tₙ₋₁, tₙ and tₙ₊₁. This step is optional. It allows certain measurement errors to be corrected.

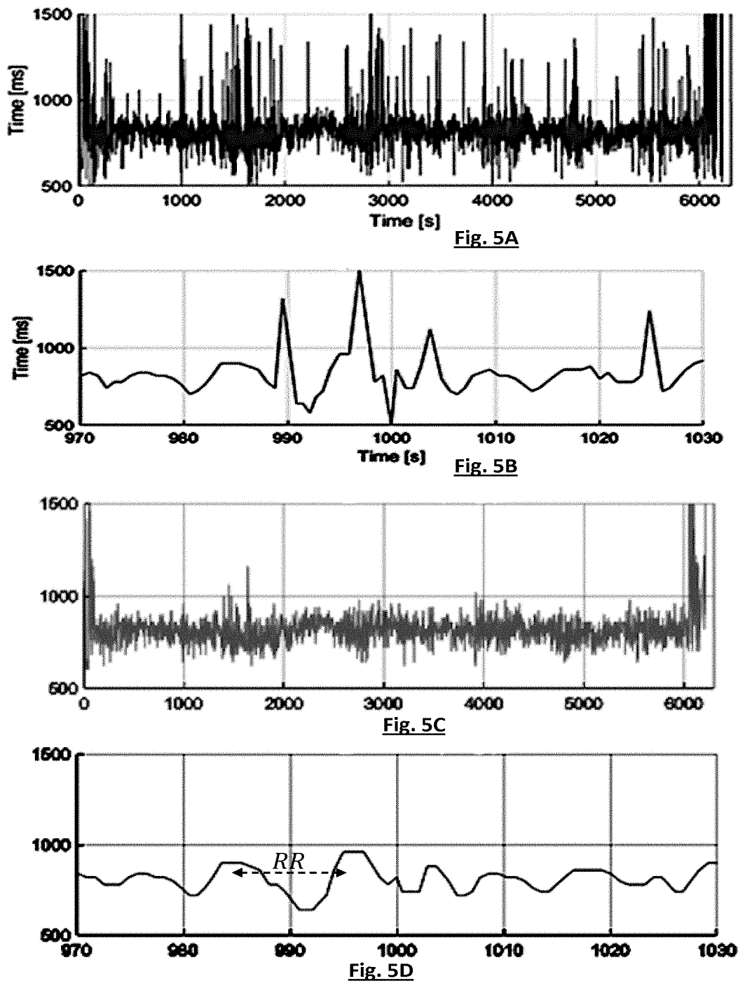

FIG. 5A shows the heart period (y-axis—units: ms) as a function of time (x-axis—units: s), obtained by implementing the method described above on the PPG signal shown in FIG. 2A, prior to smoothing. FIG. 5B is a detail of FIG. 5A, showing the heart period corresponding to the PPG signal shown in FIG. 2B.

FIG. 5C (same units as FIG. 5A) results from application of smoothing, such as described with reference to step 170, to the time-domain variation in the heart period shown in FIG. 5A. FIG. 5D is a detail of FIG. 5C, this detail having been established on the basis of the PPG signal shown in FIG. 2B. The periodic fluctuations observed in FIG. 5B correspond to the breathing rate RR. Thus, the method allows not only heart period (or heart rate) to be estimated, but also breathing rate, as a function of time, to be estimated. Furthermore, it allows this to be done with a simple device, worn at the end of a finger or able to be integrated into a mass-market watch.

The method described with reference to steps 100 to 170 may be implemented with simultaneous use of various spectral bands, for example centered on the green (preferred spectral band), and/or the red and/or the infrared. This allows an estimation of heart rate and optionally of breathing rate RR to be obtained independently in various spectral bands. The estimations in each spectral band may then be combined. The estimation considered to be closest to an estimation at a preceding time may also be selected. According to one possibility, the spectral band is selected depending on the spectral power of the frequency-domain expression FFT(g_{AC}) at the dominant frequency f. For example, it is possible to compute, in each spectral band, a ratio between the spectral power of the frequency-domain expression FFT(g_{AC}) at the dominant frequency f and the spectral power of all of the frequencies at which FFT(g_{AC}) is determined. The spectral band for which the ratio is highest is retained.

According to one variant, in step 120, the frequency-domain expression FFT_{f}(g_{AC}) for the pulsed component is obtained with an overlap of 50% of two successive time windows At. This allows a given characteristic time tₙ of the pulsed component g_{AC} to be identified twice, respectively on the basis of frequency-domain expressions FFT_{f}(g_{AC}) formed in two successive time windows. The average of the detected characteristic times may be considered, or only the characteristic times detected twice t₀ may be selected. This improves the robustness of the estimation.

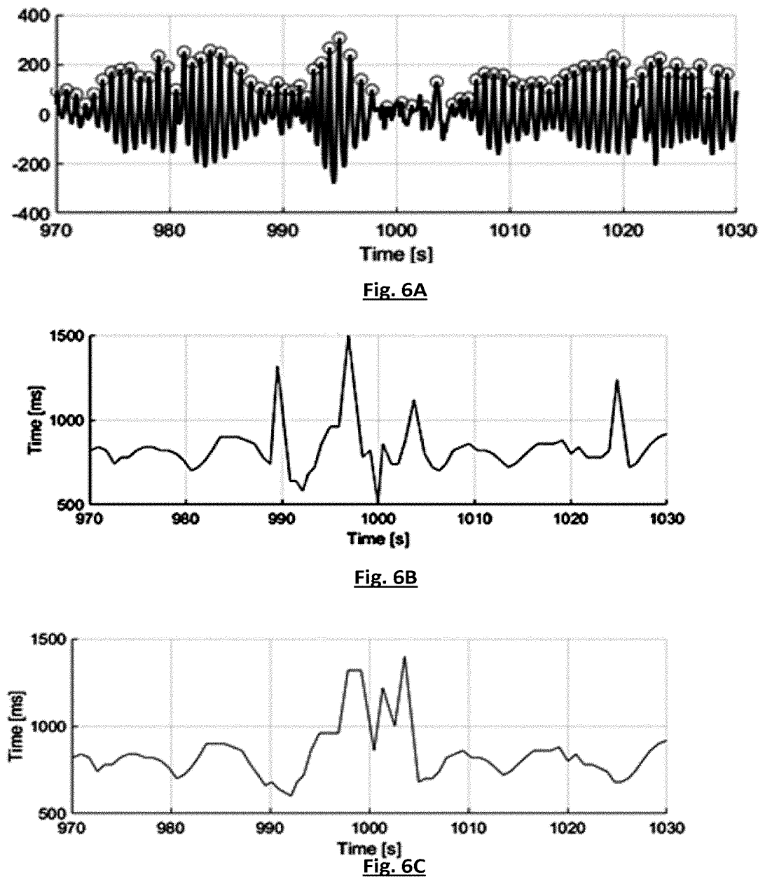

According to one variant, in step 140, the sinusoidal function sin_{f} is deformed using a dynamic-time-warping (DTW) algorithm. The sinusoidal function sin_{f} is thus modified so as to optimize a match with the pulsed component g_{AC}. According to this variant, the characteristic times t′ₙ of the sinusoidal function sin_{f} are considered to be coincident with the characteristic times tₙ of the pulsed component g_{AC}. FIG. 6A shows an example of selection of characteristic times tₙ on the pulsed component. FIGS. 6B and 6C show, respectively, the heart periods resulting:
from step 140, such as described with reference to FIG. 5B;
from the variant of step 140, implementing the DTW algorithm.

In FIG. 6A, the x-axis corresponds to time (units: seconds) and the y-axis corresponds to value of the pulsed component (arbitrary units). In FIGS. 6B and 6C, the x-axis corresponds to time (units: seconds). The y-axis corresponds to heart period: units milliseconds.

It may be seen that there is a certain similarity in the estimation of heart period, despite certain disparities. One drawback related to the DTW algorithm is a larger memory and a higher consumption than in step 140 described above.

According to one variant, a validity test may be carried out on each estimation of heart period IBI(tₙ) or of heart rate HR(tₙ). It may be a question of verifying that the heart period (or heart rate) is comprised in a validity interval. When it is a question of heart rate, the validity interval may be comprised between 40 and 200 beats per minute (bpm). Alternatively or in addition, the validity test consists in verifying that the inter-beat interval (or heart rate) is comprised in a predetermined interval about the inter-beat interval (or heart rate) estimated at a preceding time tₙ₋₁. Depending on the validity test, a validity indicator may be assigned to each estimation IBI (tₙ) or HR(tₙ) at the time tₙ.

According to one possibility, the method may comprise a step 180 of characterizing cardiac activity. To do this, the characteristic times tₙ resulting from step 160 are added to the measurement function g or to the pulsed component g_{AC}. A baseline BL is drawn between the value of the function g or of the pulsed component g_{AC}, at each characteristic time tₙ. The measurement function g, or its pulsed component g_{AC}, is then subtracted from the baseline, so as to obtain a characterization function h. The characterization function comprises lobes, each lobe representing a volume of blood between two successive heartbeats. Each lobe extends between two successive characteristic times tₙ, tₙ₊₁. This allows a more thorough characterization of cardiac activity, by way of the area and/or shape of each lobe. Preferably, this step is implemented when the characteristic times tₙ, correspond to maxima or minima of the measurement function g or on the pulsed component g_{AC}.

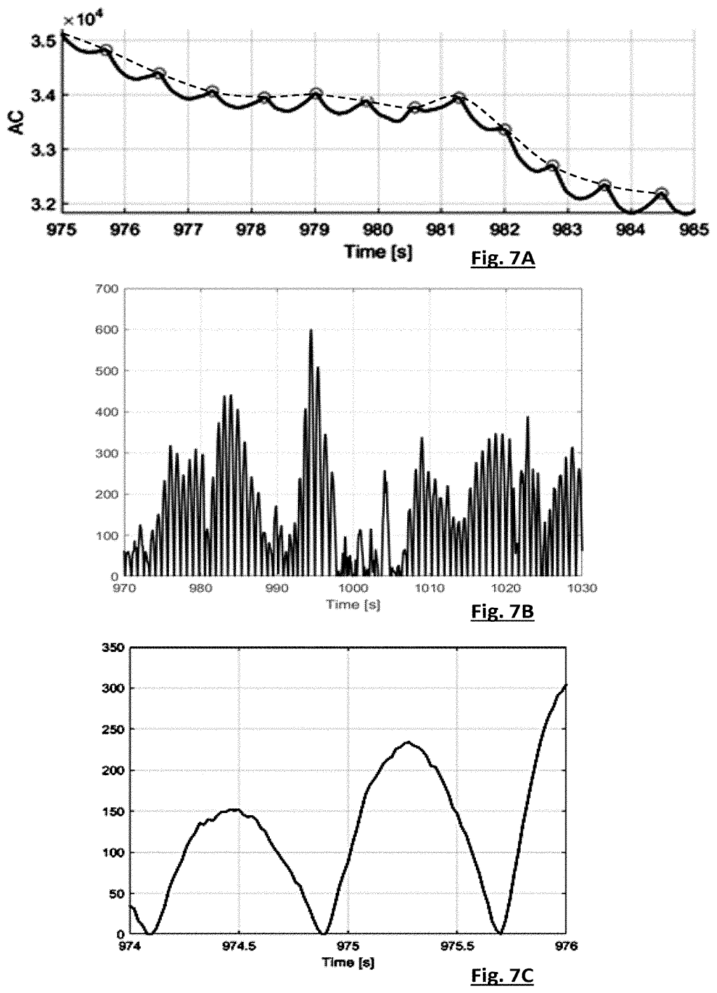

FIGS. 7A, 7B and 7C respectively show:
a detail of the measurement function described with reference to FIG. 2A (solid line), and a baseline BL passing through each characteristic time (dashed line);
a characterization function h, such that h=BL−g;
a detail of the characterization function h, showing two separate lobes: it will be understood that the characterization function h allows more thorough information as regards the cardiac activity of the user to be accessed.

In FIGS. 7A, 7B and 7C, the units of the y-axes are arbitrary units. The x-axis corresponds to time (units: seconds).

Although described with reference to an optical device 1, the invention may be applied to other types of detectors, for example to an acoustic detector, in particular one working in the ultrasound domain. It is known that cardiac echography may also allow information on pulsed variations in volumes of blood to be accessed. The invention may be applied on the basis of an echography signal, of a Doppler-echography signal for example. The device comprises at least one ultrasonic source 10′ and at least one acoustic sensor 20′. In practice, generally transducers that act both as source and detector are employed. The detected time-domain function g may be an acoustic amplitude signal or an acoustic phase signal or an acoustic frequency signal (spectrogram). Each of these signals comprises a periodic pulsed component g_{AC}, the period of which depends on heart rate. Steps 110 to 170, or even 180, described above with regard to an optical signal, may be applied, in the same way, to extract characteristic times tₙ of the pulsed component g_{AC}, so as to estimate heart rate or breathing rate.

The invention may also be applied to fetal echography. In this case, each source and each ultrasonic detector is applied against the fetus, but does not make direct contact with the latter, but rather makes contact with the belly of the mother, the latter forming a propagation medium between the device and the fetus.

The invention may also be applied to a device such as a tonometer comprising a pressure sensor applied in contact with a bodily region.

The invention may also be applied to measurements of the flow rate of air exhaled or inhaled by a user, the objective being to characterize a respiratory activity. The flow rate of air comprises a pulsed component g_{AC}, the period of which depends on the breathing rate of the user. Steps 110 to 170, or even 180, described above 30 with regard to an optical signal, may be applied, in the same way, to extract characteristic times tₙ of the pulsed component g_{AC}, so as to estimate breathing rate and/or characterize respiratory activity.

The invention may also be applied, more generally, to a measurement of any physiological parameter that varies periodically under the effect of a cardiac or respiratory activity, and in particular when the time-domain variation in the physiological parameter may be considered to be sinusoidal.

The method is relatively simple to implement. It is parameterized by:
the sampling frequency of the signals detected by the detector;
the duration Δt of the time window used to compute a frequency-domain expression for the pulsed component g_{AC} of the measurement function g ;
the duration Δt of the time intervals in which characteristic times of the pulsed component are sought on the basis of the characteristic times of the sinusoidal function sin_{f}.

The method is simple to implement and may be integrated into portable devices, for example watches or portable medical equipment.

1. A method for determining a frequency or period of a time-domain variation in a physiological parameter, the physiological parameter varying periodically as a function of time, under the effect of a cardiac activity or respiratory activity of a user, the method comprising:
a) applying an optical device against a bodily region of the user, the optical device comprising :
a light source arranged to emit light toward the bodily region;
a photodetector, arranged to detect an intensity of light emitted by the light source and having propagated through the bodily region;
illuminating the bodily region with the light source;
and measuring the intensity detected by the photodetector at various times ;
b) on the basis of the detected intensities, obtaining a measurement function, which corresponds to a time-domain variation in detected intensity;
c) obtaining a pulsed component of the measurement function, the pulsed component being a periodic function the period of which depends on the cardiac activity or respiratory activity;
d) on the basis of the pulsed component, determining heart rate or breathing rate;
wherein d) comprises:
di) obtaining a frequency-domain expression for the pulsed component;
dii) selecting a dominant frequency of the frequency-domain expression;
diii) filtering the frequency-domain expression at the dominant frequency, so as to obtain a filtered frequency-domain expression;
div) on the basis of the filtered frequency-domain expression, obtaining a sinusoidal time-domain function having the dominant frequency;
dv) taking into account a selection condition and selecting characteristic times of the sinusoidal time-domain function resulting from div), each characteristic time corresponding to a time at which said sinusoidal time-domain function meets the selection condition;
dvi) on the basis of the characteristic times of the sinusoidal time-domain function, selecting characteristic times of the pulsed component, each characteristic time of the pulsed component corresponding to a time at which said pulsed component meets the selection condition;
dvii) determining the frequency or period of the time-domain variation in the physiological parameter on the basis of the characteristic times of the pulsed component resulting from dvi).

2. The method of claim 1, wherein:
diii) comprises carrying out bandpass filtering on the frequency-domain expression resulting from di), in a passband containing the dominant frequency selected in dii), so as to obtain a filtered frequency-domain expression;
div) comprises computing a time-domain expression for the filtered frequency-domain expression, the computed time-domain expression corresponding to the sinusoidal function.

3. The method of claim 1, wherein:
in dv), the selection condition is reaching an extremum, the extremum either being a maximum, or a minimum, each characteristic time being one extremum of the sinusoidal function;
in dvi), each characteristic time is one extremum of the pulsed component.

4. The method of claim 1, wherein step dvi) comprises:
defining a time interval about each characteristic time selected on the basis of the sinusoidal time-domain function, the time interval being shorter than one period of the sinusoidal time-domain function;
in each time interval, selecting a time at which the pulsed component meets the selection condition.

5. The method of in claim 1, wherein:
step div) implements a dynamic-time-warping algorithm, so as to optimize a match between the sinusoidal function and the pulsed component;
in dvi), the characteristic times of the pulsed component correspond to the characteristic times of the sinusoidal function.

6. The method of claim 1, wherein step dvii) comprises estimating a heart rate or period.

7. The method of claim 6, wherein the heart rate or heart period is estimated on the basis of lengths of time separating successive characteristic times of the pulsed com ponent.

8. The method of claim 7, wherein dvii) comprises:
computing a time-domain variation in the heart rate or period;
determining at least one period of the time-domain variation in the heart rate or period;
estimating a breathing rate or period on the basis of the period of said time-domain variation.

9. The method of claim 8, wherein step dvii) comprises applying smoothing to the time-domain variation in the heart rate, prior to determining the breathing rate or period.

10. The method of claim 1, wherein the selection condition taken into account in dv) and dvi) is reaching an extremum, the method comprising a step of characterizing cardiac or respiratory activity, comprising:
forming a baseline, passing through the time-domain measurement function or the pulsed component at each characteristic time resulting from dvi);
obtaining a characterization function, by subtracting the time-domain measurement function or the pulsed component from the baseline, the characterization function comprising lobes, each lobe extending between two successive characteristic times;
characterizing cardiac or respiratory activity on the basis of a shape or of an area of the lobes of the characterization function.

11. A device for determining a frequency or period of a time-domain variation in a physiological parameter, the physiological parameter varying periodically as a function of time, under the effect of a cardiac activity or respiratory activity of a user, the device comprising:
an optical device, configured to be applied against a bodily region of the user, the optical device comprising :
a light source arranged to emit light toward the bodily region;
a photodetector, arranged to detect an intensity of light emitted by the light source and having propagated through the bodily region;
a processing unit, configured to implement steps b) to d) of a method of claim 1, on the basis of light intensities detected by the photodetector at the various times.
